(19) United States
(12) Patent Application Publication
(10) Pub. No.: US 2019/0336788 A1
(43) Pub. Date: Nov. 7, 2019

(54) LIGHT RADIATOR AND LIGHT SHIELDING MEMBER

(71) Applicant: SHARP KABUSHIKI KAISHA, Sakai City, Osaka (JP)

(72) Inventors: JUN MORI, Sakai City, Osaka (JP); HITOSHI AOKI, Sakai City, Osaka (JP); MASUMI MAEGAWA, Sakai City, Osaka (JP)

(73) Assignee: SHARP KABUSHIKI KAISHA, Sakai City, Osaka (JP)

(21) Appl. No.: 16/473,502

(22) PCT Filed: Dec. 11, 2017
(86) PCT No.: PCT/JP2017/044358

§ 371 Date: Jun. 25, 2019

(30) Foreign Application Priority Data
Dec. 28, 2016 (JP) 2016-256287

(51) Int. Cl. A61N 5/06

(52) U.S. Cl. CPC A61N 5/0616; A61N 2005/0658; A61N 2005/005; A61N 2005/0666; A61N 2005/0667

(57) ABSTRACT

Light can be radiated onto a desired region of skin in a reliable and simple manner while preventing the radiation of light onto regions other than the desired region. A light radiator is provided with: a light source that radiates light onto skin; a light shielding member that is arranged between the skin and the light source; and a flexible base on which the light source and the light shielding member are mounted, in which an opening that corresponds to a specific region of the skin can be formed in the light shielding member, and the light is radiated onto the specific region through the opening.

### TECHNICAL FIELD

The present invention relates to a light radiator and a light shielding member used in light irradiation therapy, beauty treatments, and the like.

### BACKGROUND ART

Photodynamic therapy (PDT) is a method of treatment in which light of a specific wavelength is radiated onto a photosensitizer that has an affinity for abnormal cells or a tumor, thereby causing a chemical reaction that produces a reactive oxygen or the like, and the sterilizing power thereof causes the abnormal cells or the tumor to undergo necrosis. This method of treatment does not harm normal cells, and has therefore been attracting much attention recently from the viewpoint of QOL (quality of life).

PDT is used for a variety of purposes including beauty treatments, pain relief, and the treatment of diseases such as neonatal jaundice, psoriasis, and acne; for example, preen light and blue-white light are used for treating neonatal jaundice, ultraviolet light is used for treating psoriasis, and blue light, red light, and yellow light are used for treating acne. In this way, a light source that radiates light of an appropriate wavelength according to the treatment purpose is used when PDT is carried out.

In recent years, lasers have become commonplace as light sources used for PDT. Examples of reasons therefor are that lasers have monochromatic light and are able to effectively excite a photosensitizer having a narrow absorption band, lasers have a high light intensity density, lasers are able to generate pulsed light, and so on. However, laser light is ordinarily spot light, has a narrow range in which radiation is possible, and is not suitable for the treatment of a skin disease or the like.

Furthermore, a case has recently been reported in which there was success in treating methicillin-resistant Staphylococcus aureus (MRSA) infected skin ulcers with PDT employing whole body administration of 5-aminolevulinic acid (ALA), which is a natural amino acid, and LED light having a wavelength of 410 nm.

ALA is a precursor of porphyrin compounds in the heme biosynthetic pathway, and is itself not photosensitizing. Physiologically, when a certain amount of heme is produced, ALA biosynthesis is inhibited by a negative feedback mechanism. However, if exogenous ALA is administered excessively, the negative feedback mechanism is abolished, and ferrochelatase which is a rate-limiting enzyme in home biosynthesis is depleted, and an endogenous porphyrin compound, protoporphyrin IX (PpIX) in particular, is accumulated in large quantities in cells. This PpIX is used as a photosensitizer in PDT employing ALA and LED light. This method of treatment does not generate new resistant bacteria, and is therefore anticipated as a new method of treatment for bacterial infection in modern medicine in which it is difficult to treat resistant bacteria.

To popularize PDT in which LED light is employed such as that mentioned above, it is necessary to realize a light irradiation device with which treatment light can be uniformly radiated onto affected parts having various three-dimensional shapes and sizes, and with which preferably there is little or no radiating of treatment light outside the affected parts.

In the case of conventionally used light sources such as excimer lamps and arc lamps, an affected part is arranged with there being a fixed distance to a fixed light source and treatment light is radiated. However, when these light sources are used, the irradiation area is too large and treatment light is also radiated onto normal sites other than the affected part, and therefore there is concern that various side effects may occur in the normal sites. Consequently, a shielding countermeasure for preventing the radiation of treatment light onto normal sites is additionally required, and treatment takes time and effort. For example, in a case where a disease that is affecting part of the face is to be treated, an eye mask (blindfold) is required to protect the eyes, which are normal sites. In addition, a mask that exposes only the affected part of the face is also required in order to protect the normal sites of the face.

Furthermore, the patient has to maintain an immobile posture for a loner period of time with the body in a constrained state for the purpose of the treatment, and there is an excessive burden on the body and fatigue accumulates. In addition, in a case where the affected part is a site having a curved surface such as part of an arm or part of a foot, for example, with an apparatus that employs a lamp-type light source, the patient may be forced into an unreasonable posture depending on whether the front surface, the rear surface, or the side of the site is to be irradiated.

Furthermore, the irradiation intensity is different for each site making up the affected area having a curved surface, depending on the angle and distance of the affected area to the apparatus employing the lamp-type light source, and therefore there are cases where it is difficult for treatment light of a uniform irradiation intensity to be radiated onto the entire affected area. Furthermore, apparatuses that employ lamp-type light sources are large and have many accessories such as power sources and cooling devices, and therefore require a large space for installation and the cost therefor is also high.

In order to solve each of the aforementioned problems, several techniques have been proposed with which treatment light can be radiated with the affected part being directly covered. For example, PTL 1 discloses an irradiation device that has LEDs serving as Light emitting sources arranged in plurality on a flexible substrate, and is able to radiate light while being wound around the affected part. Furthermore, PTL 2 discloses a light irradiation device that has LEDs serving as light emitting sources arranged on a flexible substrate and a light transmitting material arranged between the affected part and the LEDs, and is thereby able to radiate light emitted by the LEDs onto the affected part.

Furthermore, PTL 3 discloses white tape used for a phototherapy device that removes colored cellular tissue of the skin by irradiating the skin surface with laser light, pulsed high-luminance white light, or the like. The white tape can be peelably adhered to the skin surface, and can be perforated according to the size, shape, and number of affected areas of the skin.

### CITATION LIST

### Patent Literature

PTL 1: International Publication No. 2001/014012 (published on Mar. 1, 2001)

PTL 2: International Publication No. 2012/023086 (published on Feb. 23, 2012)

PTL 3: Japanese Unexamined Patent Application Publication “Japanese Unexamined Patent Application Publication No. 2005-319210 (published on Nov. 17, 2005)”

### SUMMARY OF INVENTION

### Technical Problem

However, in the irradiation device disclosed in PTL 1, the light source and a fixing implement that fixes the light source to the affected part are integrated, and it is therefore necessary for irradiation devices of different sizes to be separately made according to the treatment site such as the torso, arms, or thighs. Furthermore, in a case where the surface area of the treatment site is small, a considerably large area of skin including the periphery of the treatment site is irradiated with light. Furthermore, it is necessary to sterilize the light source and the fixing implement for each treatment, which takes time and effort. In addition, a device that circulates cooling water for cooling the light source is additionally required, which increases the cost.

Furthermore, the light irradiation device disclosed in PTL 2 uses a light source independently from the affected area when LED light is radiated, and PTL 2 does not disclose a technique for fixing the light source to the affected area. In the configuration of the aforementioned light irradiation device, there is a risk that the patient may touch the light source during treatment and cause the light source to malfunction or move the position thereof. In addition, the white tape disclosed in PTL 3 requires additional work for copying the size of the affected area, and such work takes time.

An aspect of the present invention has been devised in consideration of the aforementioned problems, and the purpose thereof is to realize a device that is capable of stably obtaining a desired light irradiation effect, in a safe and simple manner with respect to a site of the body that does not necessarily require a fixing implement such as an arm or a leg, for example.

### Solution to Problem

In order to solve the aforementioned problems, a light radiator according to an aspect of the present invention is a light radiator for radiating light onto a specific region of the skin of an irradiation target organism, provided with: a light source that radiates the light onto the skin; a light shielding member that is arranged between the skin and the light source, and thereby shields regions other than the specific region of the skin from the light; and a flexible base on which the light source and the light shielding member are mounted, in which for an opening that corresponds to the specific region can be formed in the light shielding member, and the light is radiated onto the specific region through the opening.

In order to solve the aforementioned problems, a light shielding member according to an aspect of the present invention is a light shielding member that is arranged between the skin of an irradiation target organism and a light source that radiates light onto the skin, and thereby shields regions other than a specific region of the skin from the light, in which two or more removable parts that can be removed from the light shielding member are formed in the light shielding member, and an opening that corresponds to the specific region is formed by removing any one or more removable parts out of the two or more removable parts from the light shielding member.

### Advantageous Effects of Invention

According to the light radiator and the light shielding member in an aspect of the present invention, light irradiation can be carried out safely and with a reduced burden an the irradiation target organism. Furthermore, light can be radiated onto a desired region of the skin in a reliable and simple manner while preventing the radiation of light onto regions other than the desired region.

### BRIEF DESCRIPTION OF DRAWINGS

FIG. 1 is a schematic view depicting the configuration of the front surface of a phototherapy device according to embodiment 1 of the present invention.

FIG. 2 is a schematic view depicting the configuration of the rear surface of the phototherapy device according to embodiment 1 of the present invention.

FIG. 3(a) is a cross-sectional schematic view depicting the configuration of the phototherapy device according to embodiment 1 of the present invention, and 3(b) is a cross-sectional schematic view depicting a state in which a first light shielding part has been peeled off, in the phototherapy device according to embodiment 1 of the present invention.

FIG. 4(a) is a side schematic view depicting a state in which a light radiating module according to embodiment 1 of the present invention has been wound around an acrylic rod, and FIG. 4(b) is a front schematic view depicting the state in which the light radiating module according to embodiment 1 of the present invention has been wound around the acrylic rod.

FIG. 5 is a cross-sectional schematic view depicting the configuration of a tight shielding member according to embodiment 1 of the present invention.

FIGS. 6(a) to (e) are schematic views depicting examples of a light shielding member forming part of a phototherapy device according to embodiment 2 of the present invention.

FIG. 7 is a schematic view depicting the configuration of the front surface of a phototherapy device according to embodiment 3 of the present invention.

FIG. 8 is a schematic view depicting the configuration of the rear surface of the phototherapy device according to embodiment 3 of the present invention.

FIG. 9 is a cross-sectional schematic view depicting a first example of the configuration of the phototherapy device according to embodiment 3 of the present invention.

FIG. 10 is a cross-sectional schematic view depicting a second example of the configuration of the phototherapy device according to embodiment of the present invention.

FIG. 11 is a cross-sectional schematic view depicting the configuration of a phototherapy device according to embodiment 4 of the present invention.

FIGS. 12(a) to (f) are explanatory diagrams depicting a first example of a method for using the phototherapy device according to embodiment 4 of the present invention.

FIGS. 13(a) to (h) are explanatory diagrams depicting a second example of a method for using the phototherapy device according to embodiment 4 of the present invention.

FIG. 14 is a cross-sectional schematic view depicting the configuration of a phototherapy device according to embodiment 5 of the present invention.

FIG. 15 is a schematic view depicting the configuration of a phototherapy device according to embodiment 6 of the present invention.

FIG. 16 is a cross-sectional schematic view depicting the configuration of a light shielding member according to embodiment 7 of the present invention.

FIG. 17 is a plan schematic view depicting the configuration of the light radiating module according to embodiment 1 of the present invention.

FIG. 18(a) is a cross-sectional schematic view depicting the positions of the phototherapy device and LED chips according to embodiment 1 of the present invention, and FIG. 18(b) is a schematic view depicting the positions of the phototherapy device and the LED chips according to embodiment 1 of the present invention.

FIG. 19 is a schematic view depicting the positions of the light shielding member, the LED chips, and protective resin in the phototherapy device according to embodiment 1 of the present invention.

FIG. 20 is a schematic view depicting the positions of the light radiating module, the protective resin, and a spacer in the phototherapy device according to embodiment 1 of the present invention.

FIG. 21 is a cross-sectional schematic view depicting the configuration of a phototherapy device according to embodiment 8 of the present invention.

FIG. 22 is a cross-sectional schematic view depicting the configuration of a phototherapy device according to embodiment 9 of the present invention.

FIG. 23 is a cross-sectional schematic view depicting the configuration of a phototherapy device according to embodiment 10 of the present invention.

FIG. 24 is a cross-sectional schematic view depicting the configuration of a phototherapy device according to embodiment 11 of the present invention.

FIG. 25 is a cross-sectional schematic view depicting the configuration of a modified example of the phototherapy device according to embodiment 11 of the present invention.

FIG. 26 is a cross-sectional schematic view depicting the configuration of a modified example of the phototherapy device according to embodiment 11 of the present invention.

### DESCRIPTION OF EMBODIMENTS

Hereinafter, embodiments of the present invention will be described using an example case where light irradiation therapy (hereinafter, abbreviated as “phototherapy”) is carried out using the light radiator according to an aspect of the present invention for a skin disease having a relatively small area. Hereinafter, it is assumed that the aforementioned light radiator is a device in which LEDs emitting a predetermined wavelength are mounted on the front surface of a flexible substrate and the LEDs can be lit; however, it should be noted that the light radiator is not restricted thereto. For example, the light radiator according to an aspect of the present invention can also have a laser, organic EL, or the like mounted therein instead of LEDs.

Considering that treatment light having high in-plane uniformity is to be radiated onto a skin disease having a relatively small area, it should be noted that it is desirable for the light radiator according to an aspect of the present invention to be provided with a light radiating module capable of surface emission, that is, a surface emitting light source.

Furthermore, hereinafter, it, is assumed that a treatment drug is applied to the affected area (specific region on the skin) or is taken in advance, and it is assumed that LEDs and the affected area are kept at an appropriate distance in order to radiate light uniformly onto the entire affected area.

Furthermore, the drug, the light wavelength used for treatment, the specific details of the substrate, and the like do not affect the configuration of the light radiator according to an aspect of the present invention, and are therefore not described in detail hereinafter. In addition, in the present specification, an “irradiation target organism” is not restricted to a person, and an “irradiation target organism” also includes animals.

### [Embodiment 1]

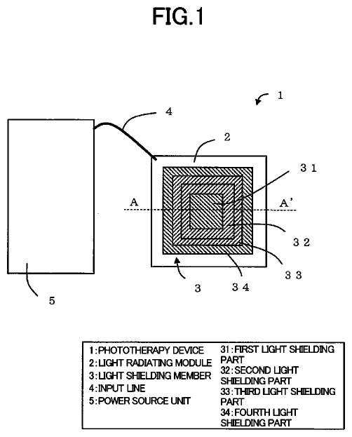

An embodiment of the present invention is as follows when described on the basis of FIGS. 1 to 5 and 17 to 20. Hereinafter, a description will be given with the front surface being the surface having mounted thereon LED chips (light sources: see FIG. 17) in a light radiating module 2, and the rear surface being the surface on the opposite side to the surface on which the LED chips 14 are mounted.

Furthermore, as depicted in FIG. 17, a plurality of LED mounting regions 17 having the LED chips 14 mounted thereon are formed in a grid form in plan view on a flexible substrate 15 (base). Pairs of adjacent LED mounting regions 17 are insulated from each other, and the pairs of LED mounting regions 17 are connected by wiring 16. It should be noted that wire-bonding connections are also included in the wiring 16.

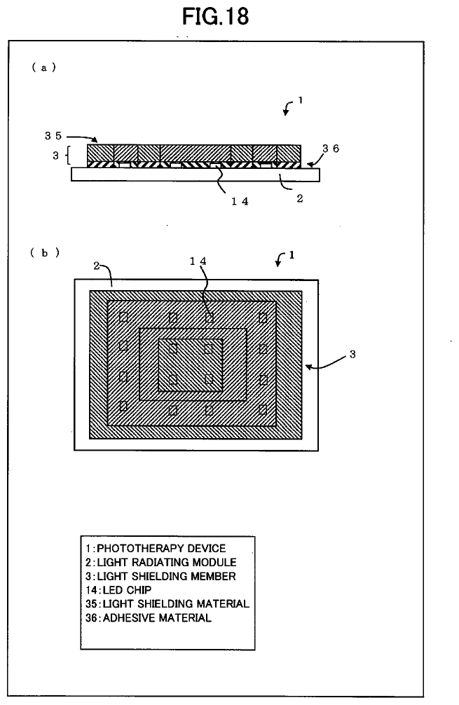

Here, the positions of a light shielding member 3 and the LED chips 14 in a phototherapy device 1 (light radiator) according to embodiment 1 of the present invention will be described using FIG. 18. As depicted in FIGS. 18(a) and (b), the LED chips 14 are mounted on the light radiating module 2, and all of the LED chips 14 are covered by the light shielding member 3. Furthermore, the light shielding member 3 is configured of a light shielding material 35 and an adhesive material 36, and the thickness of the adhesive material 36 is greater than the thickness of the LED chips 14. By adopting this kind of configuration, surface irregularities of the LED chips 14 can be reduced and the front surface of the light shielding material 35 is smooth, and it therefore becomes easy for the light shielding member 3 to be brought into contact with the skin.

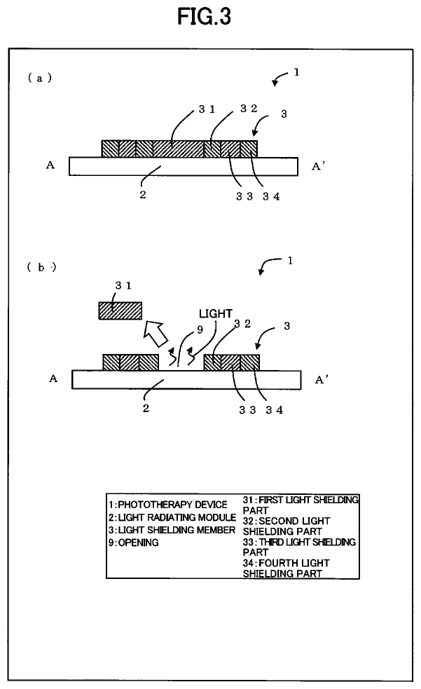

Furthermore, the outermost peripheral part of the light shielding material 35 is positioned outside the LED chips 14 at the outermost periphery. By adopting this kind of arrangement, maximum radiation efficiency can be obtained by all of the plurality of LED chips 14 in a case where the light radiation range (see FIG. 3(b): opening 9) is at the maximum.

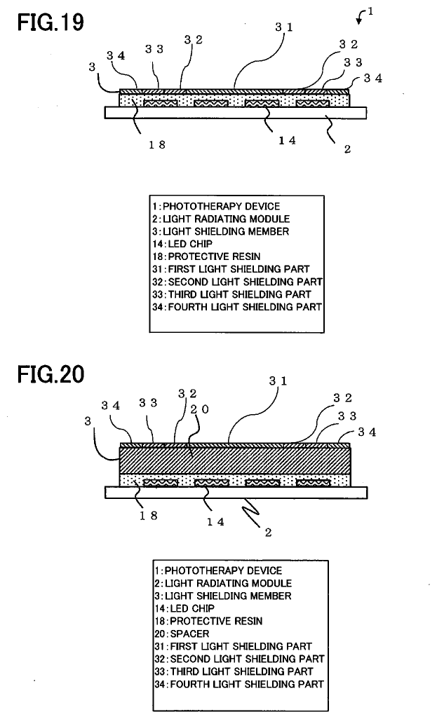

Furthermore, the positions of the light shielding member 3, the LED chips 14, and protective resin 18 in the phototherapy device 1 will be described with reference to FIG. 19. As depicted in FIG. 19, the LED chips 14 are mounted on the light radiating module 2. Furthermore, the LED chips 14 are covered by the protective resin 18, and the protective resin 18 is covered by the light shielding member 3. A material with which the protective resin 18 can be adhered is selected for the adhesive material 36.

It should be noted that a wavelength conversion member may be included in the protective resin 18. Alternatively, a resin sheet that includes a wavelength conversion member may be provided on the protective resin 18. In this case, the resin sheet that includes the wavelength conversion member is covered by the Light shielding member 3.

Furthermore, as depicted in FIG. 20, a spacer 20 having transparency with respect to the light emitted by the light radiating module 2 may be provided so that an affected part (not depicted in FIG. 20) and the light radiating module 2 are separated by a fixed distance. It is thereby possible to increase the in-plane uniformity of the light emitted by the light radiating module 2.

In addition, a wavelength conversion member may be included in the protective resin 18, or a resin sheet that includes a wavelength conversion member may be provided on the protective resin 18. Alternatively, a wavelength conversion member may be included in the spacer 20, or a resin sheet that includes a wavelength conversion member may be additionally provided on the spacer 20.

### <Configuration of Phototherapy Device>

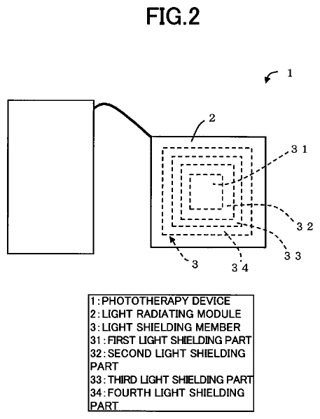

The configuration of the phototherapy device 1 according to embodiment 1 of the present invention will be described with reference to FIG. 1 to 5. FIG. 1 is a schematic view depicting the configuration of the front surface of the phototherapy device 1. FIG. 2 is a schematic view depicting the configuration of the rear surface of the phototherapy device 1. FIG. 3(a) is a cross-sectional schematic view depicting the configuration of the phototherapy device 1, and FIG. 3(b) is a cross-sectional schematic view depicting a state in which a first light shielding part has been peeled off, in the phototherapy device according to embodiment 1 of the present invention. It should be noted that FIG. 3 corresponds to a cross-sectional view along line A-A′ of the phototherapy device 1 depicted in FIGS. 1 and 2.

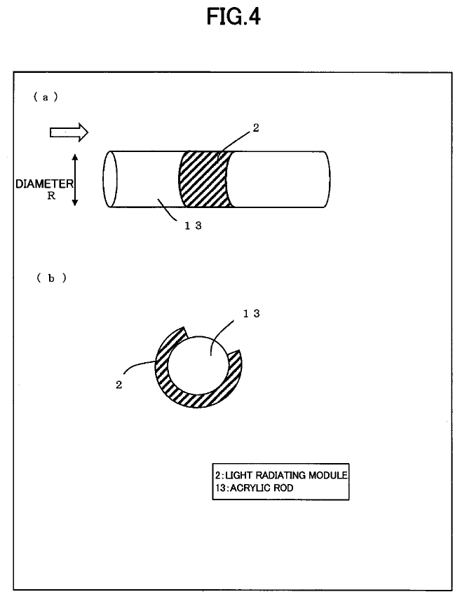

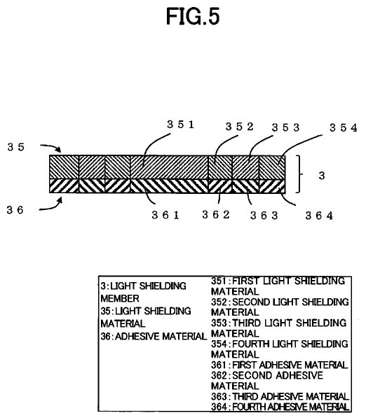

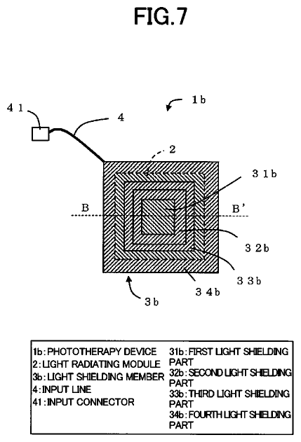

Furthermore, FIG. 4(a) is a side schematic view depicting a state in which the light, radiating module 2 has been wound around an acrylic rod 13, and FIG. 4(b) is a front schematic view depicting the state in which the light radiating module 2 has been wound around the acrylic rod 13. FIG. 5 is a cross-sectional schematic view depicting the configuration of the light shielding member 3. FIG. 7 is a plan schematic view depicting the configuration of the light radiating module 2.

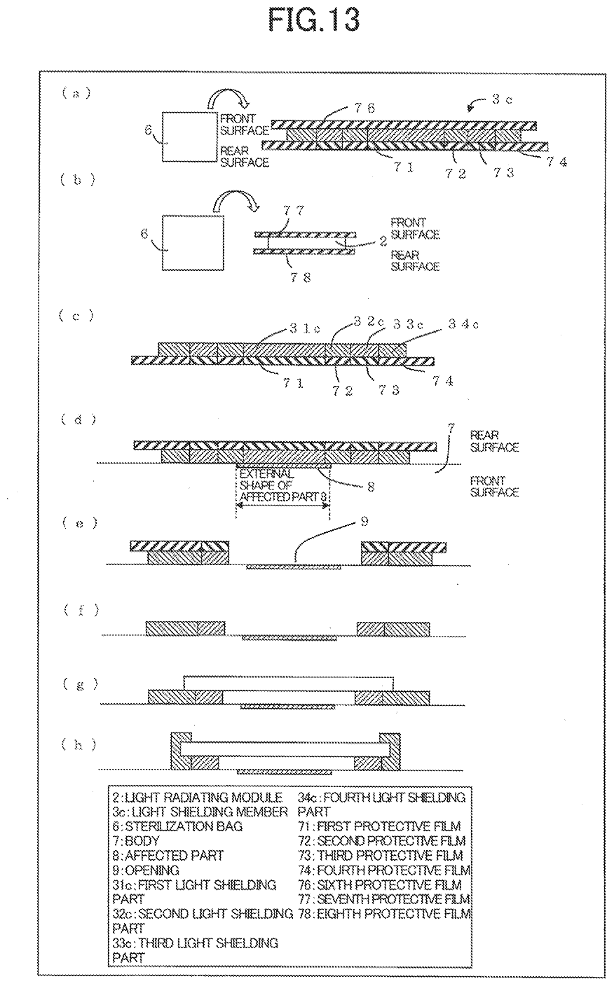

The phototherapy device 1 is a device for carrying out phototherapy on a skin disease of an irradiation target organism (not depicted) by radiating LED light (light) onto the affected part 8 (see FIG. 13 and the like) of the irradiation target organism. As depicted in FIGS. 1 to 3, in the phototherapy device 1, the light shielding member 3 is provided on the front surface of the light radiating module 2, and the light radiating module 2 and a power source unit 5 are connected by an input line 4.

### (Light Radiating Module)

The light radiating module 2 is a sheet-shaped member that has a substantially square shape in plan view, in which the LED chips 14 are mounted on the flexible substrate 15 and the flexible substrate 15 and the LED chips 14 are integrally formed. It should be noted that the light radiating module 2 may have LEDs, laser chips, or laser packages integrated with a flexible substrate, or may have a combination of organic EL, LEDs, or lasers and a light guide plate, a diffusion plate, or the like. In addition, regarding the wavelength of LED light, a suitable wavelength can be selected as appropriate in accordance with the intended phototherapy and the extent of the skin disease.

Since the sites of the irradiation target organism have surfaces that are not flat, it is assumed that LED light is to be radiated onto an affected part 8 having a surface that is not flat, and so the light radiating module 2 is made to be flexible. Specifically, as depicted in FIGS. 4(a) and (b), the light radiating module 2 is flexible to the extent of being able to be wound around the acrylic rod 13 while being in close contact with the side surface thereof, the acrylic rod 13 being cylindrical and having a diameter R=5 cm.

Here, it is desirable for the arrangement of the constituent components of the wiring 16 and the light radiating module 2 to be implemented in such a way that there is no loss of function due to a lighting failure, disconnection, or the like when the light radiating module 2 is wound around the aforementioned cylindrical acrylic rod 13. Furthermore, it is more desirable for the aforementioned loss of function to not occur together with it being possible for the light radiating module 2 to be able to be wound around a cylindrical acrylic rod 13 having a diameter of 1 cm.

It should be noted that the phototherapy device I may be provided with a piece of glass, a film substrate, a bandage, or a cloth instead of the aforementioned flexible substrate 15, and the LED chips 14 may be mounted on these bases. In other words, the phototherapy device 1 does not necessarily have to be provided with a light radiating module 2 in which the LED chips 14 and the flexible substrate 15 are integrated, and it is sufficient to be provided with a flexible base on which there are mounted some kind of light sources such as the LED chips 14.

### (Light Shielding Member)

The light shielding member 3 is a sheet-shaped member that has a substantially square shape in plan view, and is arranged between the skin of the irradiation target organism and the LED chips 14 to thereby shield normal sites other than the affected part 8 (regions other than a specific region of the skin) from LED light. As depicted in FIGS. 1 to 3, a first light shielding part 31, a second light shielding part 32, a third light shielding part 33, and a fourth light shielding part 34 (first to fourth light shielding parts 31, 32, 33, and 34: removable parts) are formed in the light shielding member 3.

The light shielding member 3 is arranged in close contact with the front surface of the light radiating module 2. Furthermore, the thickness of the light shielding member 3 is slightly less than the thickness of the light radiating module 2, and the size of the plan view of the light shielding member 3 is less than the size of the plan view of the light radiating module 2. In other words, there is a region that is not covered by the light shielding member 3, on the front surface of the light radiating module

The first light shielding part 31 is a sheet-shaped member that has a substantially square shape in plan view and is formed furthest inside the light shielding member 3 in plan view from among the first to fourth light shielding parts 31, 32, 33, and 34. The second to fourth light shielding parts 32, 33, and 34 are sheet-shaped members that have rectangular O-shapes in plan view, and are each arranged surrounding the first light shielding part 31 and increasingly nearer to the edge of the light radiating module 2 in the above-mentioned order. Specifically, an opening 9 can be formed by peeling off the first light shielding part 31, as depicted in FIG. 3(b).

One or more of the first to fourth light shielding parts 31, 32, 33, and 34 can be peeled off (removed) from the light radiating module 2 in accordance with the size/shape of the affected part 8, and openings 9 of various sizes/shapes can be formed depending on the type/number of light shielding parts that are peeled off. In other words, the opening 9 is formed corresponding to the size/shape of the affected part 8, and LED light is radiated onto the affected part 8 through this opening 9.

If the fourth light shielding part 34 is peeled off, there is no longer a portion that blocks the LED light (treatment light) emitted from the light radiating module 2. Therefore, from the viewpoint of protecting normal sites from irradiation with LED it is more preferable if the fourth light shielding part 34 is fixed to the light radiating module 2 so that the fourth light shielding part 34 cannot be peeled off.

In the present embodiment, a description has been given regarding an example in which the four light shielding parts of the first light shielding part 31, the second light shielding part 32, the third light shielding part 33, and the fourth light shielding part 34 are formed in the light shielding member 3, but it should be noted that it is not necessary for four light shielding parts to be formed in the light shielding member 3. However, it goes without saying that it is possible to handle the various sizes of affected parts to a greater extent by forming more than four light shielding parts. Furthermore, it is not necessarily required for the first light shielding part 31, the second light shielding part 32, the third light shielding part 33, and the fourth light shielding part 34 to be peeled off in this order.

In addition, the material used for the first to fourth light shielding parts 31, 32, 33, and 34 is assumed to be a composite material such as a light reflecting material (aluminum or the like), a light absorbing material (light shielding film or the like), or a biocompatible adhesive; however, there is not restriction to these composite materials.

Furthermore, the light shielding member 3 is configured from the light shielding material 35 and the adhesive material 36 as depicted in FIG. 5. The light shielding material 35 has the effect of: weakening the intensity of the LED light emitted by the light radiating module 2 to a substantially safe level, and, specifically, the surface at the side opposing the light radiating module 2 is able to carry out reflection, refraction, absorption, and the like with respect to the aforementioned LED light. For a substantially safe level, reference canal be made to Japanese Industrial Standards JISC7550 (Photobiological Safety of Lamps and Lamp Systems), JISC6802 (Safety of Laser Products), or the like. The light shielding material 35 and the light radiating module 2 are adhered with the adhesive material 36, thereby fixing the light shielding member 3 to the light radiating module 2. It should be noted that, when LED light is incident on the light shielding member 3, it is desirable for the transmitted light to be less than 30% of the incident light.

The light shielding material 35 is configured of a first light shielding material 351, a second light shielding material 352, a third light shielding material 353, and a fourth light shielding material 354, and the adhesive material 36 is configured of a first adhesive material 361, a second adhesive material 362, a third adhesive material 363, and a fourth adhesive material 364. Furthermore, the first light shielding part 31 is configured of the first light shielding material 351 and the first adhesive material 361, the second light shielding part 32 is configured of the second light shielding material 352 and the second adhesive material 362, the third light shielding part 33 is configured of the third light shielding material 353 and the third adhesive material 363, and the fourth light shielding part 34 is configured of the fourth light shielding material 354 and the fourth adhesive material 364.

In addition, it is desirable for the thickness of the light shielding material 35 to be greater than the thickness of the adhesive material 36, and is desirably of the order of 0.1 to 10 mm. If the light shielding member 3 is thicker than necessary, it becomes difficult for the light shielding member 3 to be in close contact with an affected part having a surface that is not flat. Meanwhile, if the light shielding member 3 is thinner than necessary, the intensity and light shielding effect and so forth thereof are hindered.

Here, it is desirable for the light shielding member 3 to have so-called biocompatibility for close contact with the skin of the irradiation target organism. In other words, it is desirable for the front surface of the light shielding member 3 (the surface that comes into close contact with the skin of the irradiation target organism) to be protected by a protective film, protective paper, or the like until the phototherapy device 1 is used, from the viewpoint, of preventing infection.

Furthermore, it is desirable for the front surface of the light shielding member 3 to have adhesive properties. By having adhesive properties, the phototherapy device 1 can be attached to the affected part 8 or another site of the irradiation target organism, and the phototherapy device 1 can be prevented from deviating from the affected part or the like when the irradiation target organism has moved. In this case, the front surface of the light shielding member 3 exposes a surface having adhesive properties, and there is a risk of dust or the like adhering to the front surface. From the viewpoint of avoiding this risk, it is desirable for the front surface of the light shielding member 3 to be protected by a protective film, protective paper, or the like until the phototherapy device 1 is used.

Furthermore, when any one or more of the first to fourth light shielding par 31, 32, 33, and 34 is peeled off to form the opening 9, it is feasible that the medical practitioner or the irradiation target organism (a person in this case) may touch the light shielding member 3. Consequently, it is desirable for the front surface of the light shielding member 3 to be further protected by a protective film, protective paper, or the like in addition to the aforementioned protective film or the like from the viewpoint of preventing infection. That is, it is more desirable for the front surface of the light shielding member 3 to be protected by at least two protective films, pieces of protective paper, or the like.

In addition, in a protective film, protective paper, or the like affixed to the rear surface of the light shielding member 3 (the surface at the side opposing the light radiating module 2), perforations may be formed in a form conforming with each of the first to fourth shielding parts 31, 32, 33, and 34. Alternatively, protective films, protective paper, or the like having forms conforming respectively with the openings 9 formed by peeling off any one or more of the first to fourth light shielding parts 31, 32, 33, and 34 may be superposed on the rear surface of the light shielding member 3.

Furthermore, after any one or more of the first to fourth light shielding parts 31, 32, 33, and 34 has been peeled off, an adhesive agent or an arbitrary transmissive material that is substantially transparent with respect to LED light, for example, having a transmittance of 80% or more, desirably 90% or more, may remain on the light radiating module 2. Thus, the aforementioned four light shielding parts are no longer peeled off directly from the front surface of the light radiating module 2, and, as a result, it is possible to reduce damage to the front surface of the light radiating module 2. Furthermore, for example, after the aforementioned four light shielding parts have been peeled off, a film-like member having an arbitrary filter effect may remain on the front surface of the light radiating module 2. It thereby becomes possible to eliminate light that is harmful to the irradiation target organism.

Furthermore, in FIG. 5, the surface heights of the first light shielding material 351, the second light shielding material 352, the third light shielding material 353, and the fourth light shielding material 354 are the same. If these surface heights are the same, the distance from the affected part to the light radiating module 2 is the same when any one or more of the aforementioned four light shielding materials has been peeled off, which is therefore convenient.

Furthermore, by changing the thickness of the light shielding member 3 as appropriate in accordance with each site of the light shielding member 3, when the phototherapy device 1 is mounted on an affected part having a surface that is not flat, it is possible for the light radiating module 2 to not come into contact with the affected part. By doing so, the phototherapy device 1 is suitable for a case where treatment is to be carried out on a skin disease where it is not possible to touch the affected part or a skin disease that is painful.

Furthermore, it is desirable for the light shielding member 3 to have insulating properties. Since the light shielding member 3 comes into contact with the skin of the irradiation target organism, phototherapy can be carried out safely by causing the light shielding member 3 to have insulating properties. In addition, it is desirable for the light shielding member 3 to have thermal insulation properties. In a case where the phototherapy device 1 generates heat due to the radiation of LED light or the like, that heat is shielded from the skin of the irradiation target organism by the light shielding member 3 being made to have thermal insulation properties, and phototherapy can be carried out comfortably without the person feeling unpleasant heat in the case where the irradiation target organism is a person.

### (Input Line and Power Source Unit)

The power source unit 5 supplies power to the light radiating module 2 via the input line 4. It should be noted that it is desirable for sterilization processing to be carried out since there is a possibility of the front surface of the light shielding member 3 coming into contact with the skin of the irradiation target organism. In addition, it is desirable for sterilization processing to be carried out since there is a possibility of the light radiating module 2 and the input line 4 also coming into contact with the skin of the irradiation target organism. In particular, the light shielding member 3 should not be reused among different irradiation target organisms also from the viewpoint of preventing infection, and is desirably used and disposed upon each single use. From the viewpoint of thoroughly preventing infection, it is more desirable for the light radiating module 2 and the input line 4 to also be used and disposed upon each single use.

### [Embodiment 2]

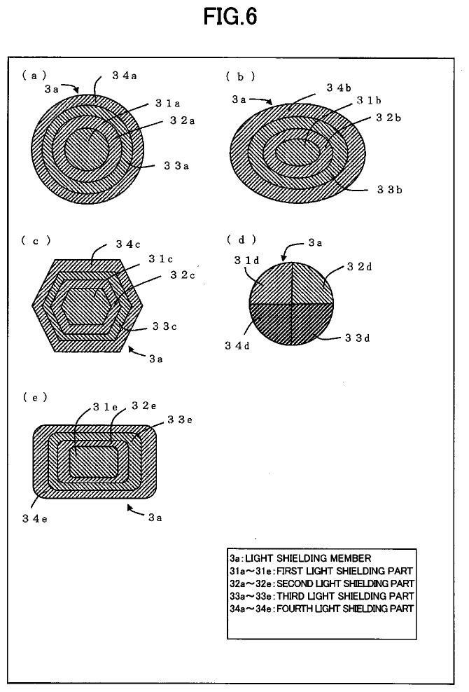

Another embodiment of the present invention is as follows when described on the basis of FIG. 6. It should be noted that, for convenience of the description, members having the same functions as the members described in the aforementioned embodiment are denoted by the same reference signs and descriptions thereof are omitted. Furthermore, in the present embodiment, differences with embodiment 1 will be described, particularly the shape of a light shielding member 3a. FIGS. 6(a) to (e) are schematic views depicting examples of the light shielding member 3a forming part of a phototherapy device 1a according to embodiment of the present invention. It should be noted that the phototherapy device 1a is obtained by replacing the light shielding member 3 of the phototherapy device 1 with the light shielding member 3a and is therefore not depicted.

### <Examples of Shape of Light Shielding Member>

Examples of the shape of the light shielding member 3a forming part of the phototherapy device 1a according to embodiment 2 of the present invention will be described with reference to FIGS. 6(a) to (e). First, the light shielding member 3a forming part of the phototherapy device 1a may be a sheet-shaped member having a circular shape in plan view, such as that depicted in FIG. 6(a). The light shielding member 3a is configured of a first light shielding part 31a, a second light shielding part 32a, a third light shielding part 33a, and a fourth light shielding part 34a.

The first light shielding part 31a is a sheet-shaped member that has a circular shape in plan view and is formed furthest inside the light shielding member 3a in plan view from among the first to fourth light shielding parts 31a, 32a, 33a, and 34a. The second to fourth light shielding parts 32a, 33a, and 34a are sheet-shaped members that have O-shapes in plan view, and are each arranged surrounding the first light shielding part 31a and increasingly nearer to the edge of the light radiating module 2 in the above-mentioned order.

It is possible for an opening 9 having a circular shape/O-shape that corresponds to the size/shape of the affected part 8 to be formed by peeling off one or more of the first to fourth light shielding parts 31a, 32a, 33a, and 34a from the light radiating module 2. It should be noted that it is not necessary for each of the aforementioned four light shielding parts to have the same center in plan view.

Next, the light shielding member 3a forming part of the phototherapy device 1a may be a sheet-shaped member having an elliptical shape in plan view, such as that depicted in FIG. 6(b). The light shielding member 3a is configured of a first light shielding part 31b, a second light shielding part 32b, a third light shielding part 33b, and a fourth light shielding part 34b. It is possible for an opening having an elliptical shape that corresponds to the size/shape of the affected part 8 to be formed by peeling off one or more of the first to fourth light shielding parts 31b, 32b, 33b, and 34b from the light radiating module 2. It should be noted that it is not necessary for each of the aforementioned four light shielding parts to have the same center in plan view.

Similarly, the light shielding member 3a forming part of the phototherapy device 1a may be a sheet-shaped member having a hexagonal shape in plan view, such as that depicted in FIG. 6(c). The light shielding member 3a is configured of a first light shielding part 31c, a second light shielding part 32c, a third light shielding part 33c, and a fourth light shielding part 34c. It is possible for an opening 9 having a hexagonal shape that corresponds to the size/shape of the affected part 8 to be formed by peeling off one or more of the first to fourth light shielding parts 31c, 32c, 33c, and 34c from the light radiating module 2. It should be noted that it is not necessary for each of the aforementioned four light shielding parts to have the same center in plan view. Furthermore, there is no restriction to a hexagon and it is also possible to form an opening 9 having a polygonal shape.

It should be noted that the aforementioned first light shielding parts 31b/31c are formed. furthest inside the light shielding member 3a in plan view from among the first to fourth light shielding parts 31b, 32b, 33b, 34b and 31c, 32c, 33c, 34c respectively.

Similarly, the light shielding member 3a forming part of the phototherapy device 1a may be a sheet-shaped member having a circular shape in plan view, such as that depicted in FIG. 6(d). The light shielding member 3a is configured of a first light shielding part 31d, a second light shielding part 32d, a third light shielding part 33d, and a fourth light shielding part 34d. The first light shielding part 31d is formed in a position that is adjacent to the second light shielding part 32d and the fourth light shielding part 34d and opposite the third light shielding part 33d by forming the four light shielding parts in this way, it is possible to form an opening 9 that corresponds to the shape of an affected part 8 that straddles a plurality of regions. Furthermore, the light shielding member 3a is not restricted to a circular shape and may be a member having a polygonal shape.

Similarly, the light shielding member 3a forming part of the phototherapy device 1a may be a sheet-shaped member having a rectangular shape in plan view with the corners having a semicircular shape that describes an arc, such as that depicted in FIG. 6(e). The light shielding member 3a is configured of a first light shielding part 31e, a second light shielding part 32e, a third light shielding part 33e, and a fourth light shielding part 34e. The first to fourth light shielding parts 31e, 32e, 33e, and 34e have at least one semicircular corner formed therein, and are able to be easily peeled off from the light radiating module 2. Furthermore, if the corners are formed with the adhesive force thereof being intentionally weakened, it is also possible to designate the corners as sites for peeling off the first to fourth light shielding parts 31e, 32e, 33e, and 34e.

It should be noted that the shape of the light shielding member 3a and the shape of the opening 9 formed in the light shielding member 3a are not restricted to the aforementioned examples. The shapes of the light shielding member 3a and the opening 9 can be formed with the aforementioned examples being combined so as to conform to the shape of the affected part 3, and, as a result, various opening patterns can be selectively made.

### [Embodiment 3]

Another embodiment of the present invention is as follows when described on the basis of FIGS. 7 to 10. It should be noted that, for convenience of the description, members having the same functions as the members described in the aforementioned embodiment are denoted by the same reference signs and descriptions thereof are omitted. Furthermore, in the present embodiment, differences with embodiments 1 and 2 will be described, particularly the configuration of a phototherapy device 1b and the shape of a light shielding member 3b.

### <Configuration of Phototherapy Device>

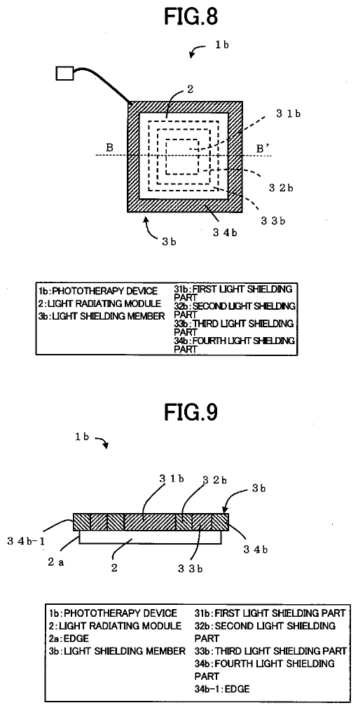

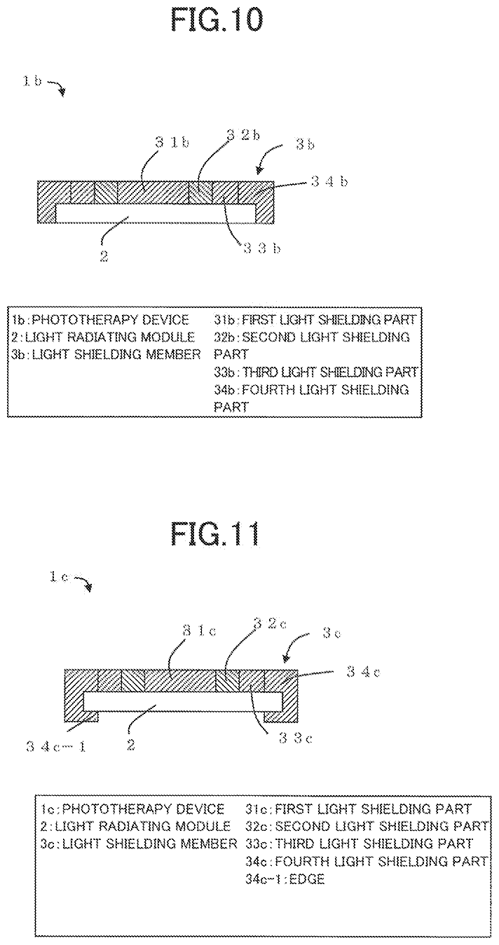

The configuration of the phototherapy device 1b according to embodiment 3 of the present invention will be described with reference to FIGS. 7 to 10. FIG. 7 is a schematic view depicting the configuration of the front surface of the phototherapy device 1b. FIG. 8 is a schematic view depicting the configuration of the rear surface of the phototherapy device 1b. FIG. 9 is a cross-sectional schematic view depicting a first example of the configuration of the phototherapy device 1b. FIG. 10 is a cross-sectional schematic view depicting a second example of the configuration of the phototherapy device 1b. It should be noted that the power source unit 5 of the phototherapy device 1b is not depicted. Furthermore, FIGS. 9 and 10 correspond to cross-sectional views along line B-B′ of the phototherapy device 1b depicted in FIG. 7. As depicted in FIG. 7, the phototherapy device 1b is provided with the light radiating module 2, an input connector 41 for connecting to the power source unit 5, and the input line 4.

As depicted in FIGS. 7 to 10, the external shape of the light shielding member 3b is configured so as to be larger than that of the light radiating module 2. Furthermore, the fourth light shielding part 34b covers the side surfaces of the light radiating module 2. That is, it is necessary for the side of the light shielding member 3 that makes contact with the light radiating module 2 to have adhesive properties. It should be noted that the portion of the light radiating module 2 that makes contact with the light shielding member 3b may have adhesive properties, or both components may have adhesive properties. Alternatively, it is also possible for known tape or the like to be used as a member for providing adhesive properties.

In an example of the configuration of the phototherapy device 1b in which the light radiating module 2 and the light shielding member 3b have quadrilateral shapes, such as that depicted in FIG. 9, an edge 34b-1 of the fourth light shielding part 34b at the outermost side protrudes from an edge 2a of the light radiating module 2; however, there is no restriction thereto. Furthermore, as depicted in FIG. 10, as another example of the configuration of the phototherapy device 1b, the fourth light shielding part 34b may be formed so as to adhere to the side surfaces of the light radiating module 2 for there to be no leakage of light.

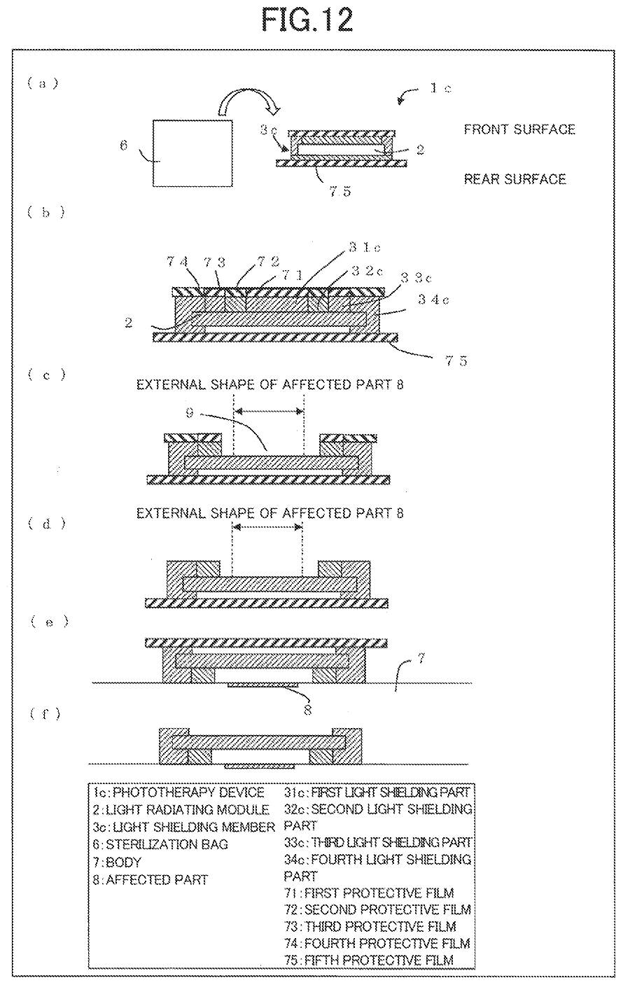

According to the phototherapy device 1b in the present embodiment, it goes without saying that it is possible to prevent LED light being radiated onto normal sites other than the affected part 8, particularly the eyes, and it is also possible to prevent LED light being radiated onto the body 7 of the medical practitioner (particularly the eyes: see FIG. 12 and the like). Furthermore, when carrying out phototherapy, it is normally necessary to separately prepare protective equipment for shielding LED light (for example, protective glasses); however, by using the phototherapy device 1b according to the present embodiment, such protective equipment is unnecessary.

### [Embodiment 4]

Another embodiment of the present invention is as follows when described on the basis of FIGS. 11 to 13. It should be noted that, for convenience of the description, members having the same functions as the members described in the aforementioned embodiment are denoted by the same reference signs and descriptions thereof are omitted. Furthermore, in the present embodiment, differences with embodiments 1, 2, and 3 will be described, particularly the configuration of a phototherapy device 1c, the shape of a light shielding member 3c, and a method for using the phototherapy device c according to the present embodiment.

### <Configuration of Phototherapy Device>

First, the configuration of the phototherapy device 1c according to embodiment 4 of the present invention will be described with reference to FIG. 11. FIG. 11 is a cross-sectional schematic view depicting the configuration of the phototherapy device 1c. As depicted in FIG. 11, the light shielding member 3c covers not only the side surfaces of the light radiating module 2 but also part of the rear surface by means of an edge 34c-1 of the fourth light shielding part 34c. In the present example in which the light radiating module L and the light shielding member 3c have quadrilateral shapes, the fourth light shielding part 34c at the outermost side protrudes; however, it should be noted that there is no restriction thereto.

Furthermore, by implementing part of the light shielding member 3c with a light transmitting material or in the form of a mesh, the light shielding member 3c is able to also perform the role of an indicator that indicates that the light radiating module 2 is lit. Due to the light shielding member 3c performing the role of an indicator, it is possible to visually recognize an operation situation in which light radiation has been reduced, and it therefore becomes easy to handle the phototherapy device 1c. In other words, since the light shielding member 3c makes it possible to visually recognize that the light radiating module 2 is lit, it is possible to reduce the risk of the patient or physician touching a light source, moving the position of a light source, or the like while light is being radiated during phototherapy.

### <Example of Method for Using Phototherapy Device>

Next, an example of a method for using the phototherapy device 1c will be described with reference to FIG. 12. FIGS. 12(a) to (f) are explanatory diagrams depicting a first example of a method for using the phototherapy device 1c. It should be noted that methods for using the phototherapy devices 1, 1a, and 1b and a phototherapy device 1d according to embodiment 5 described hereinafter are similar to the method for using the phototherapy device 1c, and therefore descriptions thereof are omitted. Furthermore, to simplify the description, in the present embodiment, a case where the phototherapy device 1 is mounted on a flat surface of the affected part 8 will be described as an example.

### (Step 1: Opening)

First, as depicted in FIG. 12(a), the sterilized phototherapy device 1c is taken out from the sterilization bag 6. Here, the light shielding member 3c is affixed to the front surface, side surfaces, and rear surface of the light radiating module 2, and a fifth protective film 75 is affixed to the rear surface of the light shielding member 3c. Furthermore, as depicted in FIG. 12(b), a first protective film 71, a second protective film 72, a third protective film 73, and a fourth protective film 74 respectively correspond to the first to fourth light shielding parts 31c, 32c, 33c, and 34c positioned on the surface of the light radiating module 2, and these four protective films are fixed to the light shielding member 3c.

### (Step 2: Forming Opening 9)

Next, as depicted in FIG. 12(c), in order to form an opening 9 that is larger than the external shape of the affected part 8 and has the smallest area outside the irradiated region, the first protective film 71 and the first light shielding part 31c are peeled off from the light radiating module 2, and then the second protective film 72 and the second light shielding part 32c are peeled off from the light radiating module 2. In the forming of the opening 9, selecting sites where protective films and light shielding parts are to be peeled off is work that is carried out by eye in accordance with the external size of the affected part 8. Furthermore, in this operation, it is desirable for the fifth protective film 75 on the rear surface of the light radiating module 2 to not be removed until this operation has been completed.

### (Step 3: Preparation for Attaching to Irradiation Target Organism)

Next, as depicted in FIG. 12(d), the third protective film 73 and the fourth protective film 74 are peeled off from the third light shielding part 33c and the fourth light shielding part 34c. It is also possible to make peeling off easy by the fourth protective film 74 being formed to have a portion that protrudes from the fourth light shielding part 34c, with the third protective film 73 being peeled off first and the fourth protective film 74 being peeled off next. It should be noted that the order in which the aforementioned protective films are removed is an example, and there is no restriction to this order.

### (Step 4: Attaching to Irradiation Target Organism)

Next, as depicted in FIG. 12 the phototherapy device to is attached to the body 7 in such a way that the affected part 8 is surrounded by the third light shielding part 33c. It is desirable for the third light shielding part 33c and the fourth light shielding part 34c to be in close contact with the body 7. Furthermore, it is desirable for the front surface of the light shielding member 3c to have adhesive properties for the phototherapy device 1c to be more reliably fixed to the body 7.

### (Step 5: Completion of Preparation)

Next, as depicted in FIG. 12(f), the fifth protective film 75 is peeled off. This operation completes the attachment, of the phototherapy device 1c to the body 7. Next, although not depicted, the input line 4, which is connected to the light radiating module 2, is connected to the power source unit 5. It should be noted that the input line 4 may be connected to the power source unit 5 by means of the input connector 41 depicted in FIGS. 7 and 8.

By going through the aforementioned steps, it is possible to decide/form a predetermined opening 9 corresponding to the shape of the affected part 8 while visually confirming the shape, and to start phototherapy.

### <Another Example of Method for Using Phototherapy Device>

Next, another example of a method for using the phototherapy device 1c will be described with reference to FIG. 13. FIGS. 13(a) to (b) are explanatory diagrams depicting a second example of a method for using the phototherapy device 1c.

### (Step 1: Opening)

First, as depicted in FIGS. 13(a) and (b), the sterilized light shielding member 3c and the light radiating module 2 are taken out from sterilization bags 6. Here, the light shielding member 3c and the light radiating module 2 are contained in separate sterilization bags 6. A sixth protective film 76 is affixed to the front surface of the light shielding member 3c, and the first protective film 71, the second protective film 72, the third protective film 73, and the fourth protective film 74 are affixed to the rear surface. Furthermore, a seventh protective film 77 is affixed to the front surface of the light radiating module 2, and an eighth protective film 78 is affixed to the rear surface.

### (Step 2: Removing Protective Films)

Next, as depicted in FIG. 13(c), the sixth protective film 76 on the front surface of the light shielding member 3c is peeled off by means of a finger or the like. It should be noted that it is desirable for the edge of the sixth protective film 76 to be formed so as to protrude from the edge of the fourth light shielding part 34c as it is therefore easy to remove the sixth protective film 76.

### (Steps 3/4: Attachment of Light Shielding Member to Irradiation Target Organism, Forming of Opening 9 Conforming With Shape of Affected Part)

Next, as depicted in FIG. 13(d), the light shielding member 3c is affixed to the body 7 of the irradiation target organism. Then, as depicted in FIG. 13(e), in order to form the smallest opening 9 that is larger than the external shape of the affected part 8, the first protective film 71 and the first light shielding part 31c are peeled off from the light radiating module 2 while observing, and next the second protective film 72 and the second light shielding part 32c are peeled off.

### (Step 5: Preparation for Attaching Light Radiating Module to Light Shielding Member)

Next, as depicted in FIG. 13(f), the seventh protective film 77 attached to the front surface of the light radiating module 2 is peeled off from the light radiating module 2. Continuing on, the third protective film 73 and the fourth protective film 74 are peeled off from the third light shielding part 33c and the fourth light shielding part 34c. When this peeling off is carried out, it is desirable for the third protective film 73 to be peeled off first, and the fourth protective film 74 having an external shape formed to be larger than the fourth light shielding part 34c to be removed next.

### (Step 6: Attaching Light Radiating Module to Light Shielding Member)

Next, as depicted in FIG. 13(g), the light radiating module 2 is attached to the light shielding member 3c. At such time, it is possible for a marking or the like showing the attachment location of the light radiating module 2 to be indicated on the light shielding member 3c. Furthermore, the attachment of the light radiating module 2 to the light shielding member 3c can be carried out more precisely by aligning the approximate center of the light shielding member 3c with the approximate center of the light radiating module 2.

### (Step 7: Completion of Preparation)

Next, as depicted in FIG. 13(h), the fourth light shielding part 34c is affixed in such a way as to go around the side surfaces and rear surface of the light radiating module 2. By using the phototherapy device 1c in this way, it is possible to visually recognize the affected part 8 through the light shielding member 3c in the aforementioned step 4, and it is therefore possible to radiate LED onto the affected part Bin a simpler and more reliable manner.

### [Embodiment 5]

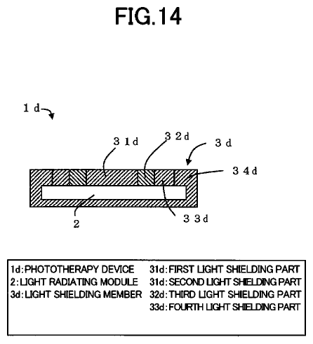

Another embodiment of the present invention is as follows when described on the basis of FIG. 14. It should be noted that, for convenience of the description, members having the same functions as the members described in the aforementioned embodiment are denoted by the same reference signs and descriptions thereof are omitted. Furthermore, in the present embodiment, differences with embodiments 1 to 4 will be described, particularly the shape of the phototherapy device 1d.

### <Configuration of Phototherapy Device>

First, the configuration of the phototherapy device 1d according to embodiment 5 of the present invention will be described with reference to 14. FIG. 11 is a cross-sectional schematic view depicting the configuration of the phototherapy device 1d. As depicted in FIG. 14, a light shielding member 3d covers not only the side surfaces of the light radiating module 2 but also the entire rear surface by means of a fourth light shielding part 34d. In the present example in which the light radiating module 2 and the light shielding member 3d have quadrilateral shapes, the fourth light shielding part 34d at the outermost side protrudes; however, it should be noted that there is no restriction thereto.

In a case where the heat dissipating properties of the fourth light shielding part 34d are low, it is expected that heat generated by the light radiating module 2 is unlikely to be emitted outside. Thus, heat generated by the light radiating module 2 may be discharged to atmosphere by using a material having high heat dissipating properties for the fourth light shielding part 34d affixed to the rear surface of the light radiating module 2, which does not come into contact with the irradiation target organism.

Furthermore, for the light shielding member 3d, the thickness does not have to be uniform, and also the material does not have to be uniform. Furthermore, similar to embodiment 4, by implementing part of the fourth light shielding part 34d with a light transmitting material or in the form of a mesh, the fourth light shielding part 34d may also be given the role of an indicator that indicates whether the light radiating module 2 is lit.

In this way, according to the phototherapy device 1d in the present embodiment, since the entire light radiating module 2 is covered by the light shielding member 3d, it is possible to further reduce the possibility of the irradiation target organism touching the light radiating module 2 even compared to the phototherapy device is according to embodiment 4.

### [Embodiment 6]

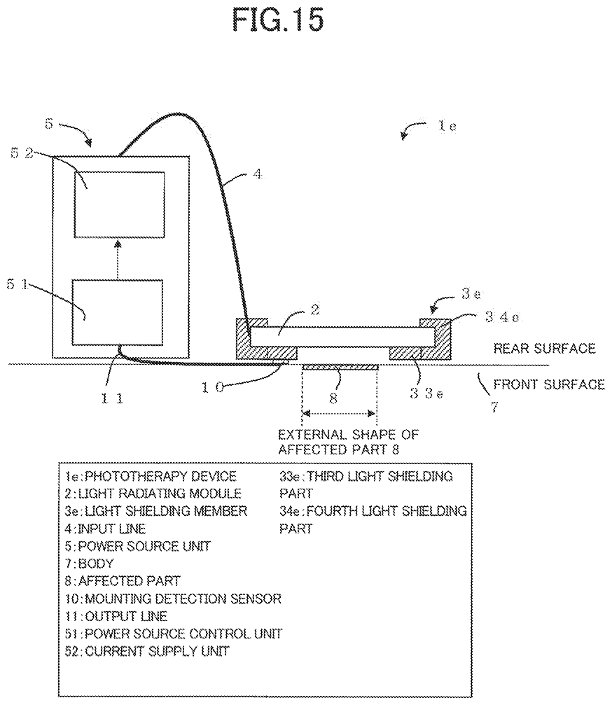

Another embodiment of the present invention is as follows when described on the basis of FIG. 15. It should be noted that, for convenience of the description, members having the same functions as the members described in the aforementioned embodiment are denoted by the same reference signs and descriptions thereof are omitted. Furthermore, in the present embodiment, differences with embodiments 1 to 5 will be described, particularly the shape of a phototherapy device 1e. The phototherapy device 1e according to the present embodiment is different from the phototherapy devices according to other embodiments in being provided with mounting detection sensor 10 (sensor) on the front surface of a light shielding member 3e.

### <Configuration of Phototherapy Device>

The configuration of the phototherapy device 1e according to embodiment 6 of the present invention will be described with reference to FIG. 15. FIG. 15 is a schematic view depicting the configuration of the phototherapy device 1e. The light shielding member 3e has a configuration in which the mounting detection sensor 10 is provided on the third light shielding part 33e, and has a configuration that is equivalent to one in which the mounting detection sensor 10 is connected to the third light shielding part 33c of the light shielding member 3c depicted in FIG. 13(b).

The mounting detection sensor 10 not only visually confirms that the light shielding member 3e and the body 7 are in close contact but also confirms such from outside, and a sensor for detecting that the phototherapy device 1e has been mounted on any site of the body 7 of the irradiation target organism, such as a pressure sensor, a tape switch, a touch sensor, a distance sensor, or the like, is sufficient.

The power source unit 5 is configured of a power source control unit 51 and a current supply unit 52, and is connected to the light radiating module 2 by the input line 4. Furthermore, the mounting detection sensor 10 is connected to the power source control unit 51 by an output line 11.

An output signal from the mounting detection sensor 10 is transmitted to the power source control unit 51 via the output line 11, and the power source control unit 51 receives the output signal and controls the current supply unit 52 on the basis of information in the output signal. Conduction control for the current supply unit 52 is carried out by, for example, the pressure that is detected in a situation where the mounting detection sensor 10 is in close contact with the body 7 being converted into current and the power source control unit 51 activating the current supply unit 52 if the current value exceeds a preset threshold value. Furthermore, the current supply unit 52 supplies current to the light radiating module 2 via the input line 4 on the basis of a control signal from the power source control unit 51.

It should be noted that it may be made possible for an operator of the phototherapy device 1e such as a medical practitioner to set the threshold value. Furthermore, the output signal from the mounting detection sensor 10 may be transmitted wirelessly. By transmitting wirelessly, the irradiation target organism can be released from the burden of being constrained for a long period of time.

In this way, by providing the phototherapy device 1e with the mounting detection sensor 10, the phototherapy device 1e is able to electrically detect being mounted on the body 7. It is therefore possible to reduce the affected part 8 being irradiated with light (treatment light) emitted from the light radiating module 2 in a state in which the phototherapy device 1e is not mounted on the body 7 or is mounted in an incomplete manner. Thus, it becomes possible to reduce uncertainty in the phototherapy effect, and the safety of the phototherapy can be increased.

### [Embodiment 7]

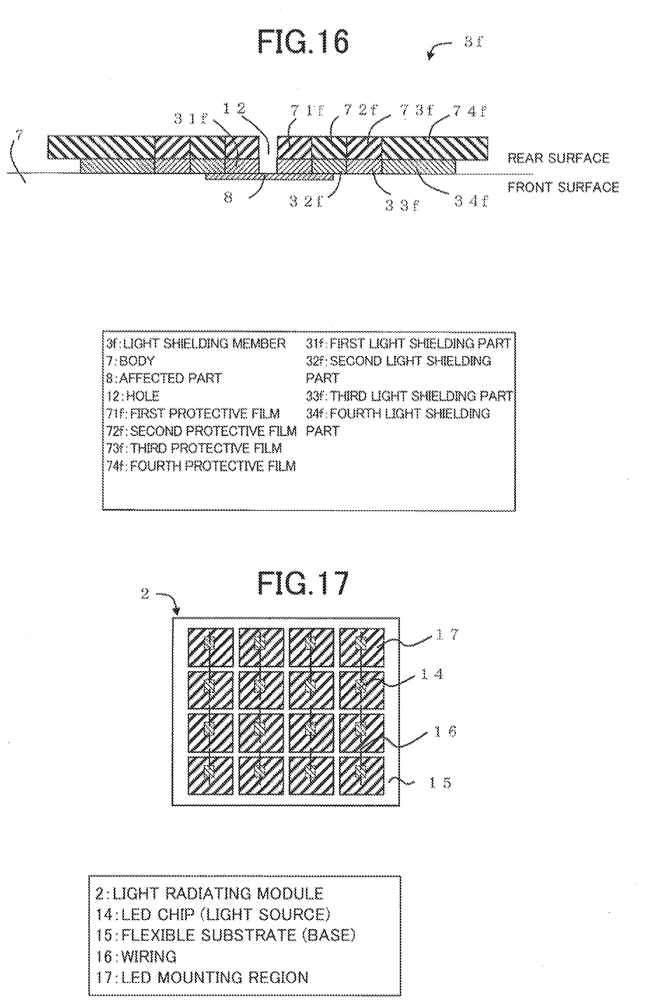

Another embodiment of the present invention is as follows when described on the basis of FIG. 16. It should be noted that, for convenience of the description, members having the same functions as the members described in the aforementioned embodiment are denoted by the same reference signs and descriptions thereof are omitted. Furthermore, in the present embodiment, differences with embodiments 1 to 6 will be described, particularly the shape of a light shielding member 3f.

<Examples of Shape of tight Shielding Member>

An example of the shape of the light shielding member 3f, which forms part of a phototherapy device if according to embodiment 6 of the present invention, will be described with reference to FIG. 16. FIG. 16 is a cross-sectional schematic view depicting the configuration of the light shielding member 3f. As depicted in FIG. 16, the light shielding member 3f is configured of first to fourth light shielding parts 31f, 32f, 33f, and 34f and first to fourth protective films 71f, 72f, 73f, and 74f.

The first protective film 71f is fixed to the first light shielding part 31f. Similarly, the second light shielding part 32f and the second protective film 72f, the third light shielding part 33f and the third protective film 73f, and the fourth light shielding part 34f and the fourth protective film 74f are respectively fixed to each other. Furthermore, regarding the light shielding member 3f, a hole 12 for visually confirming the position of the affected part 8 is formed in the approximate center of the light shielding member 3f in advance.

In this way, according to the phototherapy device 1f in the present embodiment, it becomes possible to align the approximate center of the shielding member 3f with the approximate center of the affected part 8, and therefore an opening 9 having the optimum size/shape for the affected part 8 can be formed more precisely.

### [Embodiment 8]

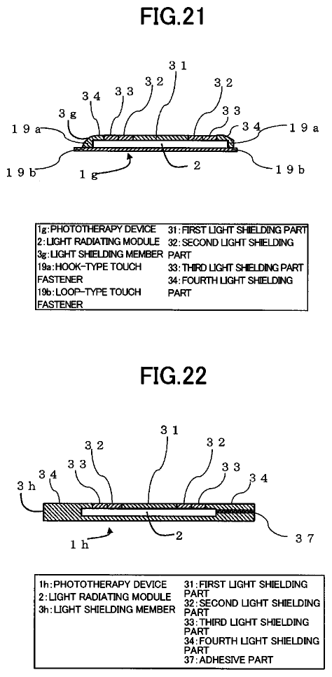

Another embodiment of the present invention is as follows when described on the basis of FIG. 21. It should be noted that, for convenience of the description, members having the same functions as the members described in the aforementioned embodiment are denoted by the same reference signs and descriptions thereof are omitted. Furthermore, in the present embodiment, differences with embodiments 1 to 7 will be described, particularly the shape of a light shielding member 3g.

As depicted in FIG. 21, the light shielding member 3g is provided in a phototherapy device 1g according to the present embodiment, and a pair of hook-type touch fasteners 19a and a pair of loop-type touch fasteners 19b are provided in part of the light shielding member 3g. Furthermore, the pair of hook-type touch fasteners 19a and the pair of loop-type touch fasteners 19b are separated between the front surface side and the rear surface side of the light radiating module 2, with the pair of hook-type touch fasteners 19a being arranged on the front surface side and the pair of loop-type touch fasteners 19b being arranged on the rear surface side.

By adhering the hook-type touch fasteners 19a and the loop-type touch fasteners 19b, the light radiating module 2 and the light shielding member 3g can be easily positioned and fixed. Furthermore, light radiated from the side surfaces of the light radiating module 2 can be efficiently shielded even in a case where the light radiating module 2 is bent in accordance with the shape of the affected part 8 (not depicted in FIG. 21).

### [Embodiment 9]

Another embodiment of the present invention is as follows when described on the basis of FIG. 22. It should be noted that, for convenience of the description, members having the same functions as the members described in the aforementioned embodiment are denoted by the same reference signs and descriptions thereof are omitted. Furthermore, in the present embodiment, differences with embodiments 1 to 8 will be described, particularly the shape of a light shielding member 3h.

As depicted in FIG. 22, the light shielding member 3h is provided in a phototherapy device 1h according to the present embodiment, and an adhesive part 37 is disposed in a site in part of the fourth light shielding part 34. By providing this adhesive part 37, by merely affixing two opposing fourth light shielding parts 34 to each other, not only it is possible for the light radiating module 2 and the light shielding member 3h to be easily positioned and fixed but it is also possible for light radiated from the light radiating module 2 to be shielded.

It should be noted that the location where the adhesive part 37 is provided is not restricted to a site in part of the fourth light shielding part 34, and the adhesive part 37 may be provided in any location as long as it is possible for the right radiating module 2 and the light shielding member 3h to be easily positioned and so forth. Furthermore, the number of adhesive parts 37 is also not restricted.

### [Embodiment 10]

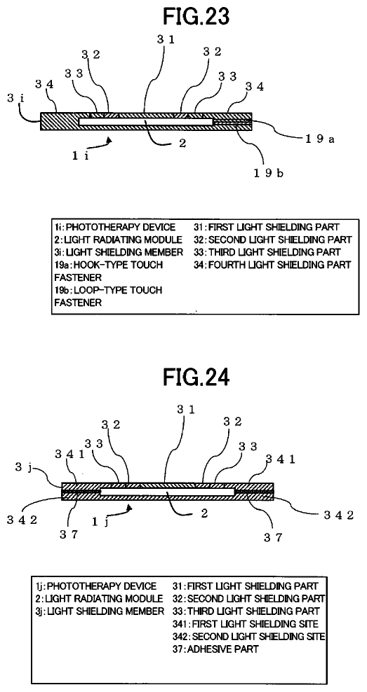

Another embodiment of the present invention is as follows when described on the basis of FIG. 23. It should be noted that, for convenience of the description, members having the same functions as the members described in the aforementioned embodiment are denoted by the same reference signs and descriptions thereof are omitted. Furthermore, in the present embodiment, differences with embodiments 1 to 9 will be described, particularly the shape of a light shielding member

As depicted in FIG. 23, the light shielding member 3i is provided in a phototherapy device 1i according to the present embodiment, a hook-type touch fastener 19a is provided in a site in part of one fourth light shielding part 34, and a loop-type touch fastener 19b is provided in a site in part of the other fourth light shielding part 34. Furthermore, the hook-type touch fastener 19a and the loop-type touch fastener 19b oppose each other, and a touch fastener is formed by adhering the hook-type touch fastener 19a and the loop-type touch fastener 9b.

Light radiated from the light radiating module 2 can be shielded in a simple manner by the aforementioned touch fastener being formed. Furthermore, the relative position of the light radiating module 2 with respect to the light shielding member 3i can be fixed.

It should be noted that the arrangement location for the touch fastener formed by the hook-type touch fastener 19a and the loop-type touch fastener 19b is not restricted to a site in part of a fourth light shielding part 34, and may be any arrangement location other than the mounting location for the light radiating module 2. Furthermore, the number of the aforementioned touch fasteners is also not restricted.

### [Embodiment 11]

Another embodiment of the present invention is as follows when described on the basis of FIGS. 24 to 26. It should be noted that, for convenience of the description, members having the same functions as the members described in the aforementioned embodiment are denoted by the same reference signs and descriptions thereof are omitted. Furthermore, in the present embodiment, differences with embodiments 1 to 10 will be described, particularly the shape of a light shielding member 3j.

As depicted in F the light shielding member 3; is provided in a phototherapy device 1j according to the present embodiment, and the fourth light shielding part 34 is configured of a first light shielding site 341 and a second light shielding site 342. The first light shielding site 341 is arranged on the front surface side of the light radiating module 2 and the second light shielding site 342 is arranged on the rear surface side.

An adhesive part 37 is provided on each of the surface of the first light shielding site 341 that opposes the second light shielding site 342 and the surface of the second light shielding site 342 that opposes the first light shielding site 341. The first light shielding site 341 and the second light shielding site 342 can be affixed by causing these two adhesive parts 37 to come into contact.

By providing the adhesive part 37 in each of the second light shielding site 342 and the first light shielding site 341 in this way, it is possible to further improve the light shielding properties of the light shielding member 3j with respect to light radiated from the side surfaces of the light radiating module 2.

It should be noted that the adhesive part 37 may be provided only on the surface of the first light shielding site 341 that opposes the second light shielding site 342, or may be provided only on the surface of the second light shielding site 342 that opposes the first light shielding site 341.

### <Modified Examples>

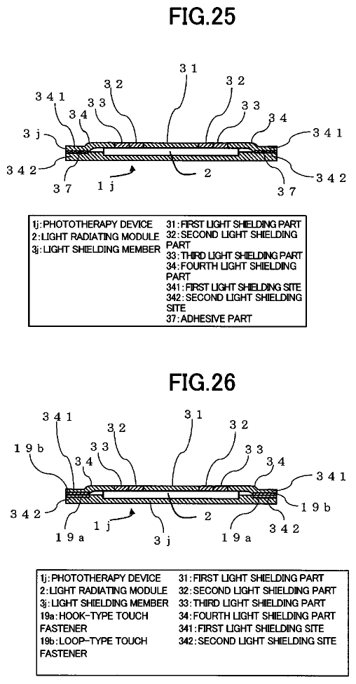

In the phototherapy device 1j according to the present embodiment, a partial space may be formed between the first light shielding site 341 and the second light shielding site 342 in a state in which the first light shielding site 341 and the second light shielding site 342 are affixed. By forming this space near the side surfaces of the light radiating module 2 as depicted in FIG. 25, it becomes possible for heat generated by the light radiating module 2 to be discharged outside the phototherapy device 1j from the space. The reliability of the phototherapy device 1j can thereby be improved. The light radiating module 2 may be cooled by filling this space with something other than air (for example, a cooling gel).

Furthermore, in the phototherapy device 11 according to the present embodiment, as well as a partial space being formed between the first light shielding site 341 and the second light shielding site 342, instead of the adhesive part 37, a pair of hook-type touch fasteners 19a may be provided at the first light shielding site 341 side and a pair of loop-type touch fasteners 19b may be provided at the second light shielding site 342 side, as depicted in FIG. 26.

By adopting this kind of configuration, light radiated from the light radiating module 2 can be shielded in a simple manner by means of touch fasteners configured of the hook-type touch fasteners 19a and the loop-type touch fasteners 19b. Furthermore, the relative position of the light radiating module 2 with respect to the light shielding member 3j can be fixed.

### [Summary]

A light radiator (light radiator 1, 1a to 1j) according to aspect 1 of the present invention is a light radiator for radiating light onto a specific region (affected part 8) of skin of an irradiation target organism, provided with: a light source that radiates the light onto the skin; a light shielding member (3, 3a to 3j) that is arranged between the skin and the light source, and thereby shields regions other than the specific region of the skin from the light; and a flexible base on which the light source and the light shielding member are mounted, in which an opening (9) that corresponds to the specific region can be formed in the light shielding member, and the light is radiated onto the specific region through the opening (9).

According to the aforementioned configuration, the light radiated from the light source is radiated onto only the specific region of the skin of the irradiation target organism, and the light is shielded by the light shielding member in regions other than the specific region. Furthermore, it is possible for the opening (9) in the light shielding member serving as a passage route for the light to be formed corresponding to the specific region. Thus, the light radiator according to an aspect of the present invention is able to reliably radiate light onto a desired region where light is intended to be radiated, while preventing the radiation of light onto regions other than the desired region.

Furthermore, according to the aforementioned configuration, there is a reduction in the risk of various side effects occurring in regions other than the desired region due to light being radiated onto regions other than the desired region, and it is therefore possible to safely radiate light onto the desired region where light is intended to be radiated.

In addition, according to the aforementioned configuration, the light source and the light shielding member are mounted on a flexible base. Consequently, even in a case where light is radiated onto a site having a curved surface such as an arm or leg of a person, for example, the light source and the light shielding member can be easily arranged in the vicinity of the site by causing the base to deform in accordance with the shape of the site. Thus, the light radiator according to an aspect of the present invention is able to radiate light in a simple manner onto a desired region where light is intended to be radiated.

In a light radiator according to aspect 2 of the present invention, in the aforementioned aspect 1, the light source is a surface emitting light source.

In a light radiator according to aspect 3 of the present invention, in the aforementioned aspect 1 or 2, LED chips (14) are formed in a grid form in plan view, as the light source, on the base.

In a light radiator (1b, 1c, 1d, 1e) according to aspect 4 of the present invention, in any of the aforementioned aspects 1 to 3, the light source and the base are integrated and form a light radiating module (2), and an edge of the light shielding member (3b, 3c, 3d, 3e) protrudes from an edge of the light radiating module.

According to the aforementioned configuration, it is possible for the skin to be reliably shielded from light by the light shielding member compared to a case where the light source and the base are not integrated. Furthermore, light that leaks out from the edge of the light radiating module is shielded by the edge of the light shielding member. Thus, it is possible to more reliably prevent light from being radiated onto regions other than the desired region where light is intended to be radiated.

In a light radiator according to aspect 5 of the present invention, in the aforementioned aspect 4, a side surface of the light radiating module is covered by the edge of the light shielding member. According to the aforementioned configuration, light that leaks out from the side surface of the light radiating module is also shielded by the edge of the light shielding member as welt as light that leaks out from the edge of the light radiating module. Thus, it is possible to prevent light more reliably/effectively from being radiated onto regions other than the desired region where light is intended to be radiated.

In a light radiator according to aspect 6 of the present invention, in the aforementioned aspect 4 or 5, a rear surface of the light radiating module is covered by part of the light shielding member.

In a light radiator according to aspect 7 of the present invention, in any of the aforementioned aspects 1 to 6, in a case where the light radiated from the light source is incident on the light shielding member, transmitted light is less than 30% of incident light.

In a light radiator (1e) according to aspect 8 of the present invention, in any of the aforementioned aspects 1 to 7, there is further provided a sensor (mounting detection sensor 10) for detecting that the light radiator has been mounted on any site of a body (7) of the irradiation target organism, and the sensor is arranged on at least part of a surface, of the light shielding member (3e), that opposes the skin.

According to the aforementioned configuration, if the light radiator according to an aspect of the present invention is mounted on any site of the body of the irradiation target organism, the sensor touches the skin at the site. Consequently, the sensor is able to reliably detect that the light radiator according to an aspect of the present invention has been mounted at the site, and it is possible to prevent light being radiated in a state in which the light radiator is not mounted or is mounted in an incomplete manner. Thus, the light radiator according to an aspect of the present invention is able to safely obtain a light radiation effect, and is able to radiate light more safely with there being a reduction in the radiation of light onto the skin of an unintended site.

In a light radiator phototherapy device 1, 1a to 1j) according to aspect 9 of the present invention, in any of the aforementioned aspects 1 to 8, the light shielding member (3, 3a to 3j) has insulating properties.

In a light radiator (phototherapy device 1c) according to aspect 10 of the present invention, in any of the aforementioned aspects 1 to 9, part of the light shielding member (3c) is formed of a material having transmissive properties with respect to the light radiated from the light source, or has a mesh form.

In a light radiator (phototherapy device 1f) according to aspect 11 of the present invention, in any of the aforementioned aspects 1 to 10, a hole (12) for visually confirming the specific region is formed in the light shielding member (3f).

In a light radiator (phototherapy device 1, 1a to 1j) according to aspect 12 of the present invention, in any of the aforementioned aspects 1 to 11, the light shielding member (3, 3a to 3j) is configured from at least a light shielding material and as adhesive material, and a thickness of the light shielding material is greater than a thickness of the adhesive material.

In a light radiator according to aspect 13 of the present invention, in any of the aforementioned aspects 1 to 12, the adhesive material (36) or a film-like member is provided between the light shielding member and the light source, and each of the adhesive material and the film-like member is transparent.

In a light radiator (phototherapy device 1f) according to aspect 14 of the present invention, in any of the aforementioned aspects 1 to 13, a mounting detection sensor (10) capable of confirming that the light shielding member and the skin are in contact is provided on the light shielding member (3f).

In a light radiator (phototherapy device 1g, 1i, 1j) according to aspect 15 of the present invention, in any of the aforementioned aspects 1 to 14, a touch fastener (hook-type touch fastener 19a, loop-type touch fastener 19b) is formed in part of the light shielding member (3g, 3i, 3j).

A light shielding member (3, 3a to 3j) according to aspect 16 of the present invention is a light shielding member that is arranged between skin of an irradiation target organism and a light source that radiates light onto the skin, and thereby shields regions other than a specific region of the skin from the light, in which two or more removable parts (first light shielding parts 31 to 34) that can be removed from the light shielding member are formed in the light shielding member, and an opening (9) that corresponds to the specific region is formed by removing any one or more removable parts out of the two or more removable parts from the light shielding member.

According to the aforementioned configuration, in the light shielding member according to an aspect of the present invention, an opening that corresponds to a desired region where light is intended to be radiated can be formed by removing any one or more removable parts from the light shielding member as appropriate in accordance with the size/shape of the desired region. Thus, by using the light shielding member according to an aspect of the present invention for radiating light onto a desired region of the skin of an irradiation target organism, it is possible for light to be reliably radiated onto the desired region while preventing the radiation of light onto regions other than the desired region.

A light shielding member according to aspect 17 of the present invention, in the aforementioned aspect 16, has insulating properties.

In a light shielding member (3c) according to aspect 18 of the present invention, in the aforementioned aspect 16 or 17, part of the light shielding member is formed of a material having transmissive properties with respect to the light radiated from the light source, or has a mesh form.

In a light shielding member (3f) according to aspect 19 of the present invention, in any of the aforementioned aspects 16 to 18, a hole (12) for visually confirming the specific region is formed in the light shielding member. According to the aforementioned configuration, by visually confirming a hole formed in the light shielding member according to an aspect of the present invention, with respect to the position of a desired region where light is intended to be radiated, it is possible for the opening to be formed more reliably in the position of the desired region. Thus, by using the light shielding member according to an aspect of the present invention for radiating light onto a desired region of the skin of an irradiation target organism, it is possible for light to be more reliably radiated onto the desired region.

A light shielding member (3, 3a to 3j) according to aspect 20 of the present invention, in any of the aforementioned aspects 16 to 19, is configured from at least a light shielding material and an adhesive material, and a thickness of the light shielding material is greater than a thickness of the adhesive material.

In a light shielding member according to aspect 21 of the present invention, in any of the aforementioned aspects 16 to 20, the adhesive material (36) or a film-like member is provided between the light shielding member and the light source, and each of the adhesive material and the film-like member is transparent.

A light shielding member (3f) according to aspect 22 of the present invention, in any of the aforementioned aspects 16 to 21, is provided with a mounting detection sensor (10) capable of confirming that the light shielding member and the skin are in contact.

In a light shielding member (3g, 3i, 3j) according to aspect 23 of the present invention, in any of the aforementioned aspects 16 to 22, a touch fastener (hook-type touch fastener 19a, loop-type touch fastener 19b) is formed in part of the light shielding member.

### Reference Signs List

1, 1a to 1j Phototherapy device (light radiator)

Light radiating module

2a Edge (edge of light radiating module)

3, 3a to 3j Light shielding member

4 Input line

5 Power source unit

6 Sterilization bag

7 Body

8 Affected part (specific region of skin of irradiation target organism)

9 Opening

10 Mounting detection sensor (sensor)

11 Output line

12 Hole

13 Acrylic rod

14 LED chip

15 Flexible substrate (base)

16 Wiring

17 LED mounting region

18 Protective resin

19a Hook-type fastener

19b Loop-type fastener

20 Spacer

31, 31a to 31f First light shielding part (removable part)

32, 32a to 32f Second light shielding part (removable part)

33, 33a to 33f Third light shielding part (removable part)

34, 34a to 34f Fourth light shielding part (removable part)

34b-1, 34c-1 Edge of fourth light shielding part (edge of light shielding member)

35 Light shielding material

36 Adhesive material

41 Input connector

51 Power source control unit

52 Current supply unit

71, 71f First protective film

72, 72f Second protective film

73, 73f Third protective film

74, 74f Fourth protective film

75 Fifth protective film

76 Sixth protective film

77 Seventh protective film

78 Eighth protective film

341 First light shielding site

342 Second light shielding site

351 First light shielding material

352 Second light shielding material

353 Third light shielding material

354 Fourth light shielding material

361 First adhesive material

362 Second adhesive material

363 Third adhesive material

364 Fourth adhesive material

1. A light radiator for radiating light onto a specific region of skin of an irradiation target organism, the light radiator comprising:
a light source that is configured to radiate the light onto the skin;
a light shielding member that is arranged between the skin and the light source, and that is configured to shield regions other than the specific region of the skin from the light; and
a flexible base on which the light source and the light shielding member are mounted,
wherein an opening that corresponds to the specific region can be formed in the light shielding member, and the light is radiated onto the specific region through the opening,
the light shielding member is configured from at least a light shielding material and an adhesive material, and
a thickness of the light shielding material is greater than a thickness of the adhesive material.

2. The light radiator according to claim 1,
wherein the light source is a surface emitting light source.

3. The light radiator according to claim 1,
wherein LED chips are formed in a grid form in plain view, as the light source, on the base.

4. The light radiator according to claim 1, wherein the light source and the base are integrated and form a light radiating module, and an edge of the light shielding member protrudes from an edge of the light radiating module.

5. The light radiator according to claim 4,
wherein a side surface of the light radiating module is covered by the edge of the light shielding member.

6. The light radiator according to claim 4,
wherein a rear surface of the light radiating module is covered by part of the light shielding member.

7. The light radiator according to claim 1,
wherein, in a case where the light radiated from the light source is incident on and transmitted through the light shielding member, transmitted light that is transmitted through the light shielding member is less than 30% of incident light that is incident on the light shielding member.

8. The light radiator according to claim 1,
further comprising a sensor for detecting that the light radiator has been mounted on any site of a body of the irradiation target organism,
wherein, in a state in which the light radiator is mounted on any site of the body, the sensor is arranged on at least part of a surface, of the light shielding member, that opposes the skin.

9. The light radiator according to claim 1,
wherein the light shielding member has insulating properties.

10. The light radiator according to claim 1,
wherein part of the light shielding member is formed of a material having transmissive properties with respect to the light radiated from the light source, or has a mesh form.

11. A light radiator for radiating light onto a specific region of skin of an irradiation target organism, the light radiator comprising:
a light source that is configured to radiate the light onto the skin;
a light shielding member that is arranged between the skin and the light source, and that is configured to shield regions other than the specific region of the skin from the light; and
a flexible base on which the light source and the light shielding member are mounted,
wherein an opening that corresponds to the specific region can be formed in the light shielding member, and the light is radiated onto the specific region through the opening, and
a hole for visually confirming the specific region is formed in the light shielding member.

12. (canceled)

13. A light radiator for radiating light onto a specific region of skin of an irradiation target organism, the light radiator comprising:
a light source that is configured to radiate the light onto the skin;
a light shielding member that is arranged between the skin and the light source, and that is configured to shield regions other than the specific region of the skin from the light; and
a flexible base on which the light source and the light shielding member are mounted,
wherein for an opening that corresponds to the specific region can be formed in the light shielding member, and the light is radiated onto the specific region through the opening,
an adhesive material or a film-like member is provided between the light shielding member and the light source, and
each of the adhesive material and the film-like member is transparent.

14. The light radiator according to claim 1,
wherein a mounting detection sensor capable of confirming that the light shielding member and the skin are in contact is provided on the light shielding member.

15. The light radiator according to claim 1,
wherein a touch fastener is formed in part of the light shielding member.

16. The light radiator according to claim 1,
wherein two or more removable parts that can be removed from the light shielding member are formed in the light shielding member, and
an opening that corresponds to the specific region is formed by removing any one or more removable parts out of the two or more removable parts from the light shielding member.

17. (canceled)

18. (canceled)

19. The light radiator according to claim 1,
wherein a hole for visually confirming the specific region is formed in the light shielding member.

20. (canceled)

21. The light radiator according to claim 1,
wherein the adhesive material or a film-like member is provided between the light shielding member and the light source, and
each of the adhesive material and the film-like member is transparent.

22. (canceled)

23. (canceled)
